(19) United States
(12) Patent Application Publication
(10) Pub. No.: US 2005/0112159 A1
(43) Pub. Date: May 26, 2005

(54) Cosmetic composition comprising at least one anionic fixing polymer and at least one amphoteric starch

(75) Inventor: Christine Dupuis, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL

(21) Appl. No.: 10/976,962

(22) Filed: Nov. 1, 2004

Related U.S. Application Data
(62) Division of application No. 08/835,360, filed on Apr. 7, 1997.

(30) Foreign Application Priority Data
Apr. 5, 1996 (FR) 96 04367

(51) Int. Cl. A61K 7/06; A61K 7/11

(52) U.S. Cl. 424/401; 424/70.13

(57) ABSTRACT

A cosmetic composition for the treatment of keratinous fibres, in particular hair, comprising at least one fixing polymer and at least one amphoteric starch and the process for treatment of keratinous fibres using said composition.

The present invention relates to a cosmetic composition for the treatment of keratinous fibres, in particular hair, comprising at least one fixing polymer and at least one amphoteric starch, and to the process for the treatment of keratinous fibres using this composition.

Hair shaping or form retention compositions containing styling polymers (fixing polymers) in their formulation generally exhibit the disadvantage of making it difficult to disentangle, restyle or brush the hair, in particular during blow-drying. Hair treated with these fixing polymers is generally harsh and has an unnatural feel.

The combination of silicone derivatives with fixing polymers is known in cosmetic compositions for form retention and/or fixing of the hairstyle. It has been found that these silicone derivatives improve the properties of disentangling, of softness and of sheen of hair treated using these compositions. However, silicone derivatives are not favourable to the styling properties of compositions containing fixing polymers.

In particular, the search is for compositions which make it possible to modify the texture of the hair, that is to say compositions which soften the hair during application on wet hair (emollience property) and which also make possible very easy disentanglement. These properties are generally achieved by cations but the hair, once dried, is rendered limp and heavy by these compositions. In other words, the hairstyle does not have body.

The aim of the present invention is thus to provide compositions which have good properties of fixation and/or of shape-retention for hair over time and which have excellent cosmetic properties, such as emollience, softness, disentangling and feel.

The inventor has now discovered, surprisingly, that by using compositions containing a fixing polymer in combination with at least one amphoteric starch in a cosmetically acceptable medium, excellent cosmetic properties, such as softness, disentangling and feel, are obtained while having synergic styling and/or fixing properties.

The fixing power of the compositions according to the invention is superior to that of compositions containing only a single one of either a fixing polymer or an amphoteric starch.

The subject of the present invention is thus a cosmetic composition for keratinous fibres comprising, in a cosmetically acceptable medium, at least one fixing polymer and at least one amphoteric starch.

In the context of the present application, cosmetic compositions for form retention of the hairstyle is understood to mean any composition having the function of temporarily fixing the shape of the hairstyle, such as, for example, styling lacquers and sprays or styling gels and foams. The fixing power of the composition denotes the ability of the composition to give the hair a cohesion such that the initial hair shaping of the hairstyle is retained. Fixing polymer is understood to mean any polymer having the function of fixing the shape of the hairstyle.

According to the present invention, the amphoteric starches and the amphoteric polymers may optionally be zwitterionic.

According to the present invention, the term “keratinous fibres” comprises the hair, the eyelashes, the eyebrows and more particularly the hair.

The amphoteric starches which can be used according to the invention contain at least one anionic group and at least one cationic group.

The at least one anionic group and at least one cationic group can be bonded to the same reactive site of the starch molecule or to different reactive sites; they are preferably bonded to the same reactive site.

The at least one anionic group can preferably be of carboxyl, phosphate or sulphate type and more preferably carboxyl type. The at least one cationic group can preferably be of primary, secondary, tertiary or quaternary amine type.

The starches which can be used according to the invention are preferably chosen from the compounds of following formulae: 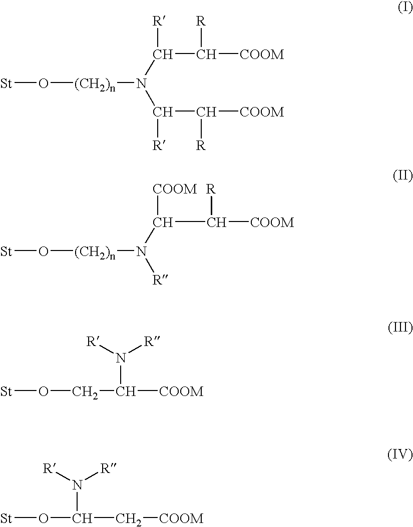
wherein:
St—O represents a starch molecule,
R independently represents a hydrogen atom or a methyl radical,
R′ independently represents a hydrogen atom, a methyl radical or a —COOH group,
n is an integer equal to 2 or 3,
M independently denotes a hydrogen atom, an alkali or alkaline-earth metal, such as Na, K or Li, NH₄, or an organic amine,
R″ represents a hydrogen atom or an alkyl radical having from 1 to 18 carbon atoms.

These compounds are in particular described in U.S. Pat. No. 5,455,340 and U.S. Pat. No. 4,017,460, the disclosures of which are specifically incorporated by reference herein.

The starch molecules can originate from any plant source of starch, such as, in particular, maize, potatoes, oats, rice, tapioca, sorghum, barley or wheat. The hydrolysates of the abovementioned starches can also be used. The starch preferably originates from potatoes.

Use is particularly made of starches of formulae (I) or (II). Use is more particularly made of starches modified by (2-chloroethyl)aminodipropionic acid, that is to say the starches of formula (I) or (II) in which R, R′, R″ and M represent a hydrogen atom and n is equal to 2.

According to the invention, any fixing polymer known per se can be used. Use may in particular be made of a fixing polymer selected from anionic, cationic, amphoteric and non-ionic polymers and their mixtures.

The fixing polymers can be used in the dissolved form or in the form of dispersions of solid polymer particles.

The fixing cationic polymers which can be used according to the present invention are preferably chosen from polymers containing primary, secondary, tertiary and/or quaternary amine groups forming part of the polymer chain or directly connected to the latter and having a molecular weight preferably ranging from approximately 500 to approximately 5,000,000 and more preferably ranging from 1000 to 3,000,000.

Mention may more particularly be made, among these polymers, of the following cationic polymers:

(1) Homopolymers or copolymers derived from acrylic or methacrylic esters or amides and preferably containing at least one of the units of following formulae: 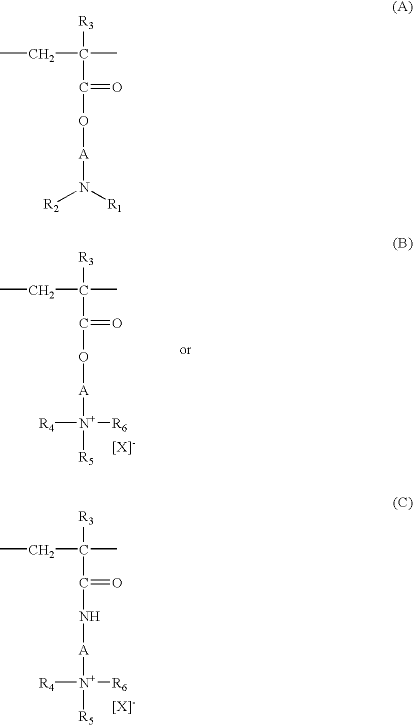
wherein:
R₃ denotes a hydrogen atom or a CH₃ radical;
A is a linear or branched alkyl group containing from 1 to 6 carbon atoms or a hydroxyalkyl group containing from 1 to 4 carbon atoms;
R₄, R₅ and R₆, independently represent an alkyl group having from 1 to 18 carbon atoms or a benzyl radical;
R₁ and R₂ independently represent hydrogen or an alkyl group having from 1 to 6 carbon atoms;
X denotes a methyl sulphate anion or a halide, such as chloride or bromide.

The copolymers of the family (1) preferably additionally contain one or a number of units deriving from comonomers which can be chosen from the family of acrylamides, methacrylamides, diacetone acrylamides, acrylamides and methacrylamides substituted on the nitrogen by lower alkyls, acrylic or methacrylic acids or their esters, vinyllactams, such as vinylpyrrolidone or vinylcaprolactam, or vinyl esters.

Thus, mention may be made, among these copolymers of the family (1), of:
copolymers of acrylamide and of dimethylaminoethyl methacrylate which is quaternized with dimethyl sulphate or with a methyl halide, such as that sold under the name HERCOFLOC by the company Hercules,
copolymers of acrylamide and of methacryloyloxy-ethyltrimethylammonium chloride described, for example, in Patent Application EP-A-080,976 and sold under the name BINA QUAT P 100 by the company Ciba-Geigy,
the copolymer of acrylamide and of methacryloyloxy-ethyltrimethylammonium methyl sulphate sold under the name RETEN by the company Hercules,
optionally quaternized vinylpyrrolidone/dialkylaminoalkyl acrylate or methacrylate copolymers, such as the products sold under the name “GAFQUAT” by the company ISP, such as, for example, “GAFQUAT 734” or “GAFQUAT 755”, or alternatively the products named “COPOLYMER 845, 958 and 937”. These polymers are described in detail in French Patents 2,077,143 and 2,393,573, the disclosures of which are specifically incorporated by reference herein,
the dimethylaminoethyl methacrylate/vinylcaprolactam/vinylpyrrolidone terpolymer, such as the product sold under the name GAFFIX VC 713 by the company ISP,
and the quaternized dimethylaminopropyl-methacrylamide/vinylpyrrolidone copolymer, such as the product sold under the name “GAFQUAT HS 100” by the company ISP.

(2) Quaternized polysaccharides, described more particularly in U.S. Pat. No. 3,589,578 and U.S. Pat. No. 4,031,370, the disclosures of which are specifically incorporated by reference herein, such as guar gums containing cationic trialkylammonium groups.

Such products are sold in particular under the trade names of JAGUAR C13 S, JAGUAR C15 and JAGUAR C 17 by the company Meyhall.

(3) Quaternary copolymers of vinylpyrrolidone and of vinylimidazole.

(4) Chitosans or their salts;

The salts which can be used are in particular chitosan acetate, lactate, glutamate, gluconate or pyrrolidonecarboxylate.

Mention may be made, among these compounds, of chitosan having a degree of deacetylation of 90.5% by weight sold under the name KYTAN CRUDE STANDARD by the company Aber Technologies or chitosan pyrrolidonecarboxylate sold under the name KYTAMER PC by the company Amerchol.

(5) Cationic cellulose derivatives, such as copolymers of cellulose or of cellulose derivatives grafted with a water-soluble monomer containing a quaternary ammonium and described in particular in U.S. Pat. No. 4,131,576, the disclosure of which is specifically incorporated by reference herein, such as hydroxyalkyl celluloses, such as hydroxymethyl, hydroxyethyl or hydroxypropyl celluloses, grafted in particular with a methacryloyl-oxyethyltrimethylammonium,methacrylamidopropyltrimethylammonium or dimethyldiallylammonium salt.

The commercialized products corresponding to this definition are more particularly the products sold under the name “CELQUAT L 200” and “CELQUAT H 100” by the Company National Starch.

The fixing anionic polymers generally used are polymers containing groups derived from carboxylic, sulphonic or phosphoric acid and have a molecular weight ranging from approximately 500 to approximately 5,000,000.

1) The carboxyl groups are introduced by unsaturated mono- or dicarboxylic acid monomers such as those corresponding to the formula: 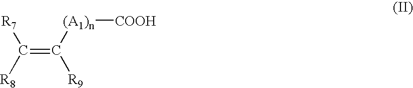
wherein:
n is an integer ranging from 0 to 10,
A₁ denotes a methylene group, optionally connected to the carbon atom of the unsaturated group, or to the neighbouring methylene group, when n is greater than 1, via a heteroatom, such as oxygen or sulphur,
R₇ denotes a hydrogen atom or a phenyl or benzyl group,
R₈ denotes a hydrogen atom or a lower alkyl or carboxyl group, and
R₉ denotes a hydrogen atom, a lower alkyl group or a —CH₂—COOH, phenyl or benzyl group.

In the abovementioned formula, a lower alkyl radical preferably denotes a group having from 1 to 4 carbon atoms and in particular methyl and ethyl.

The preferred fixing anionic polymers containing carboxyl groups according to the invention are:

A) Homo- or copolymers of acrylic or methacrylic acid or their salts and in particular the products sold under the names VERSICOL E or K by the company Allied Colloid and ULTRAHOLD by the company BASF, copolymers of acrylic acid and of acrylamide sold in the form of their sodium salt under the names RETEN 421, 423 or 425 by the company Hercules or the sodium salts of polyhydroxycarboxylic acids.

B) Copolymers of acrylic or methacrylic acid with a monoethylenic monomer, such as ethylene, styrene, vinyl esters or esters of acrylic or methacrylic acid, optionally grafted onto a polyalkylene glycol, such as polyethylene glycol, and optionally crosslinked. Such polymers are described in particular in French Patent 1,222,944 and German Patent Application 2,330,956, the disclosures of which are specifically incorporated by reference herein, the copolymers of this type containing, in their chain, an optionally N-alkylated and/or -hydroxyalkylated acrylamide unit, such as described in particular in Luxembourg Patent Applications 75370 and 75371, the disclosures of which are specifically incorporated by reference herein, or proposed under the name QUADRAMER by the Company American Cyanamid.

Mention may also be made of copolymers of acrylic acid and of C₁-C₄ alkyl methacrylate and terpolymers of vinylpyrrolidone, of acrylic acid and of C₁-C₂₀ alkyl methacrylate, for example lauryl methacrylate, such as that sold by the company ISP under the name ACRYLIDONE LM, and methacrylic acid/ethyl acrylate/tert-butyl acrylate terpolymers, such as the product sold under the name LUVIMER 100 P by the company. BASF.

C) Copolymers derived from crotonic acid, such as those containing, in their chain, vinyl acetate or propionate units and optionally other monomers, such as allyl or methallyl esters, vinyl ether or vinyl ester of a linear or branched saturated carboxylic acid containing a long hydrocarbon chain, such as those containing at least 5 carbon atoms, it being possible for these polymers optionally to be grafted and crosslinked, or alternatively a vinyl, allyl or methallyl ester of an α- or β-cyclic carboxylic acid. Such polymers are described, inter alia, in French Patents FR 1,222,944, 1,580,545, 2,265,782, 2,265,781, 1,564,110 and 2,439,798, the disclosures of which are specifically incorporated by reference herein. Commercial products coming within this class are the resins 28-29-30, 26-13-14 and 28-13-10 sold by the company National Starch.

D) Copolymers derived from monounsaturated C₄-C₈ carboxylic acids or anhydrides chosen from:

copolymers comprising (i) one or a number of maleic, fumaric or itaconic acids or anhydrides and (ii) at least one monomer chosen from vinyl esters, vinyl ethers, vinyl halides, phenylvinyl derivatives or acrylic acid and its esters, the anhydride functional groups of these copolymers optionally being monoesterified or monoamidified. Such polymers are described in particular in U.S. Pat. Nos. 2,047,398, 2,723,248 and 2,102,113 and British Patent GB 839,805, the disclosures of which are specifically incorporated by reference herein, and in particular those sold under the names GANTREZ AN or ES by the company ISP.
copolymers comprising (i) at least one of maleic, citraconic or itaconic anhydrides and (ii) at least one monomers chosen from allyl or methallyl esters, optionally containing at least one acrylamide, methacrylamide, α-olefin, acrylic or methacrylic ester, acrylic or methacrylic acid or vinylpyrrolidone groups in their chain,
   the anhydride functional groups of these copolymers optionally being monoesterified or monoamidified.
   These polymers are, for example, described in French Patents FR 2,350,384 and FR 2,357,241, the disclosures of which are specifically incorporated by reference herein.
   E) Polyacrylamides containing carboxylate groups.

The polymers comprising sulpho groups are polymers containing vinylsulpho, styrenesulpho, naphthalenesulpho or acrylamidoalkylsulpho units.

These polymers can in particular be chosen from:
salts of polyvinylsulphonic acid having a molecular weight ranging from approximately 1000 to approximately 100,000, as well as copolymers with an unsaturated comonomer, such as acrylic or methacrylic acids and their esters, as well as acrylamide or its derivatives, vinyl ethers and vinylpyrrolidone.
salts of polystyrenesulphonic acid, the sodium salts having a molecular weight ranging from approximately 500,000 to approximately 100,000 sold respectively under the names FLEXAN 500 and FLEXAN 130 by National Starch. These compounds are described in French Patent FR 2,198,719, the disclosure of which is specifically incorporated by reference herein.
salts of polyacrylamidesulphonic acids, those mentioned in U.S. Pat. No. 4,128,631, the disclosure of which is specifically incorporated by reference herein, and more particularly polyacrylamidoethylpropanesulphonic acid sold under the name COSMEDIA POLYMER HSP 1180 by Henkel.

According to the invention, the fixing anionic polymers are preferably chosen from copolymers of acrylic acid, such as the acrylic acid/ethyl acrylate/N-tert-butylacrylamide terpolymer sold under the name ULTRAHOLD STRONG by the company BASF, copolymers derived from crotonic acid, such as the vinyl acetate/vinyl tert-butylbenzoate/crotonic acid terpolymers and the crotonic acid/vinyl acetate/vinyl neododecanoate terpolymers sold under the name RESIN 28-29-30 by the company National Starch, polymers derived from maleic, fumaric or itaconic acids or anhydrides with vinyl esters, vinyl ethers, vinyl halides, phenylvinyl derivatives or acrylic acid and its esters, such as the monoesterified maleic anhydride/methyl vinyl ether copolymer sold under the name GANTREZ ES 425 by the company ISP, copolymers of methacrylic acid and of methyl methacrylate sold under the name EUDRAGIT L by the company Rohm Pharma, the copolymer of methacrylic acid and of ethyl acrylate sold under the name LUVIMER MAEX or MAE by the company BASF, the vinyl acetate/crotonic acid copolymer sold under the name LUVISET CA 66 by the company BASF and the copolymer of vinyl acetate of crotonic acid grafted with polyethylene glycol under the name ARISTOFLEX A by the company BASF.

The most particularly preferred fixing anionic polymers are chosen from the monoesterified maleic anhydride/methyl vinyl ether copolymer sold under the name GANTREZ ES 425 by the company ISP, the acrylic acid/ethyl acrylate/N-tert-butylacrylamide terpolymer sold under the name ULTRAHOLD STRONG by the company BASF, the copolymers of methacrylic acid and of methyl methacrylate sold under the name EUDRAGIT L by the company Rohm Pharma, the vinyl acetate/vinyl tert-butylbenzoate/crotonic acid terpolymers and the crotonic acid/vinyl acetate/vinyl neododecanoate terpolymers sold under the name RESIN 28-29-30 by the company National Starch, the copolymer of methacrylic acid and of ethyl acrylate sold under the name LUVIMER MAEX or MAE by the company BASF or the vinylpyrrolidone/acrylic acid/lauryl methacrylate terpolymer sold under the name ACRYLIDONE LM by the company ISP.

The fixing amphoteric polymers which can be used in accordance with the invention can be chosen from polymers containing B and C units distributed statistically in the polymer chain, where B denotes a unit deriving from a monomer containing at least one basic nitrogen atom and C denotes a unit deriving from an acidic monomer containing at least one carboxyl or sulpho groups, or alternatively B and C can denote groups deriving from zwitterionic carboxybetaine or sulphobetaine monomers;

B and C can also denote a cationic polymer chain containing primary, secondary, tertiary or quaternary amine groups, in which at least one of the amine groups carries a carboxyl or sulpho group connected via a hydrocarbon radical or alternatively B and C form part of a chain of a polymer containing an α,β-dicarboxy ethylene unit in which one of the carboxyl groups has been reacted with a polyamine containing one or a number of primary or secondary amine groups.

The more particularly preferred fixing amphoteric polymers corresponding to the definition given above are chosen from the following polymers:
1) Polymers resulting from the copolymerization of a monomer derived from a vinyl compound carrying a carboxyl group, such as more particularly acrylic acid, methacrylic acid, maleic acid or α-chloroacrylic acid, and of a basic monomer derived from a substituted vinyl compound containing at least one basic atom, such as more particularly dialkylaminoalkyl methacrylate and acrylate or dialkylaminoalkylmethacrylamide and -acrylamide. Such compounds are described in U.S. Pat. No. 3,836,537, the disclosure of which is specifically incorporated by reference herein.

(2) Polymers containing units deriving:
a) from at least one monomer chosen from acrylamides or methacrylamides substituted on the nitrogen by an alkyl radical,
b) from at least one acidic comonomer containing at least one reactive carboxyl groups, and
c) from at least one basic comonomer, such as esters containing primary, secondary, tertiary and quaternary amine substituents of acrylic and methacrylic acids and the product from the quaternization of dimethylaminoethyl methacrylate with dimethyl or diethyl sulphate.

The more particularly preferred N-substituted acrylamides or methacrylamides according to the invention are the groups in which the alkyl radicals contain from 2 to 12 carbon atoms and more particularly N-ethylacrylamide, N-tert-butylacrylamide, N-tert-octylacrylamide, N-octylacrylamide, N-decylacrylamide or N-dodecylacrylamide, and the corresponding methacrylamides.

The acidic comonomers are more particularly chosen from acrylic, methacrylic, crotonic, itaconic, maleic and fumaric acids and the alkyl monoesters, having from 1 to 4 carbon atoms, of maleic or fumaric acids or anhydrides.

The preferred basic comonomers are aminoethyl, butylaminoethyl, N,N′-dimethylaminoethyl and N-tert-butylaminoethyl methacrylates.

Use is particularly made of copolymers whose CTFA name (4th Ed., 1991) is Octylacrylamide/acrylates/butylaminoethyl methacrylate copolymer, such as the products sold under the name AMPHOMER or LOVOCRYL 47 by the company National Starch.

(3) Partially or totally alkylated and crosslinked polyaminoamides deriving from polyaminoamides of general formula:
CO—R₁₀—CO-Z (III)
wherein: R₁₀ represents a divalent radical derived from a saturated dicarboxylic acid, from an aliphatic mono- or dicarboxylic acid containing an ethylenic double bond, from an ester of a lower alkanol having from 1 to 6 carbon atoms of these acids or from a radical deriving from the reaction of any one of the said acids with a bisprimary or bissecondary amine and Z denotes a radical from a bisprimary or mono- or bissecondary polyalkylenepolyamine and preferably represents:

a) in the proportions of from 60 to 100 mol %, the radical
—NH(CH₂)ₓ—NHₚ (IV)
wherein
x=2 and p=2 or 3 or alternatively x=3 and p=2,
   this radical deriving from diethylenetriamine, triethylenetetraamine or dipropylenetriamine;

b) in the proportions of from 0 to 40 mol %, the above radical (IV),

wherein

x=2 and p=1 and which derives from ethylenediamine, or the radical deriving from piperazine:

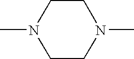

c) in the proportions of from 0 to 20 mol %, the —NH—(CH₂)₆—NH— radical deriving from hexamethylenediamine, these polyaminoamides being crosslinked by addition of a bifunctional crosslinking agent chosen from epihalohydrins, diepoxides, dianhydrides or bisunsaturated derivatives, by means of from 0.025 to 0.35 mol of crosslinking agent per amine group of the polyaminoamide, and alkylated by reaction with acrylic acid, with chloroacetic acid or with an alkanesultone, or with their salts.

The saturated carboxylic acids are preferably chosen from acids having from 6 to 10 carbon acids, such as adipic, 2,2,4-trimethyladipic and 2,4,4-trimethyladipic or terephthalic acid, acids containing an ethylenic double bond, such as, for example, acrylic, methacrylic and itaconic acids.

The alkanesultones used in the alkylation are preferably propane- or butanesultone and the salts of the alkylating agents are preferably the sodium or potassium salts.

(4) Polymers containing zwitterionic units of formula: 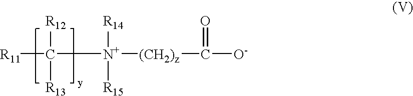
wherein:
R₁₁ denotes a polymerizable unsaturated group, such as an acrylate, methacrylate, acrylamide or methacrylamide group,
y and z represent an integer ranging from 1 to 3,
R₁₂ and R₁₃ independently represent a hydrogen atom, methyl, ethyl or propyl, and
R₁₄ and R₁₅ independently represent a hydrogen atom or an alkyl radical, so that the sum of the carbon atoms in R₁₄ and R₁₅ does not exceed 10.

The polymers comprising such units can also contain units derived from non-zwitterionic monomers, such as dimethyl- or diethylaminoethyl acrylate or methacrylate or alkyl acrylates or methacrylates, acrylamides or methacrylamides or vinyl acetate.

Mention may be made, by way of example, of the methyl methacrylate/methyidimethylcarboxymethylammonio-ethylmethacrylate copolymer, such as the product sold under the name DIAFORMER Z301 by the company Sandoz.

(5) Polymers derived from chitosan containing monomer units corresponding to the following formulae: 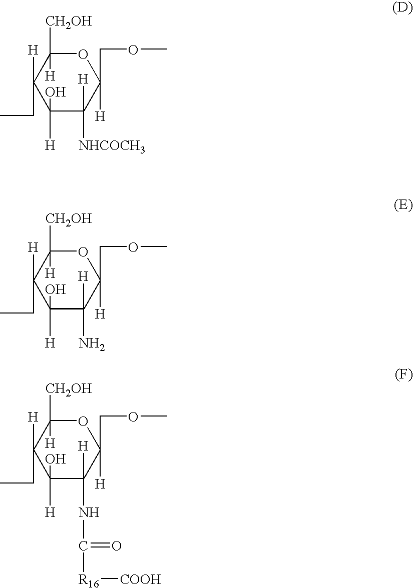
wherein:
the D unit is present in proportions ranging from 0 to 30%,
the E unit is present in proportions ranging from 5 to 50%, and
the F unit is present in proportions ranging from 30 to 90%,
it being understood that, in this F unit, R₁₆ represents a radical of formula: 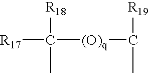
   wherein:
if q=0, R₁₇, R₁₈ and R₁₉ each independently represent a hydrogen atom, a methyl, hydroxyl, acetoxy or amino residue, a monoalkylamino residue or a dialkylamino residue, which are optionally interrupted by one or a number of nitrogen atoms and/or optionally substituted by one or a number of amino, hydroxyl, carboxyl, alkylthio or sulpho groups, or an alkylthio residue, in which the alkyl group carries an amino residue, at least one of the R₁₇ R₁₈ and R₁₉ radicals being, in this case, a hydrogen atom;
   or,
if q=1, R₁₇, R₁₈ and R₁₉ each represent a hydrogen atom, and the salts formed by these compounds with bases or acids.

(6) Polymers derived from the N-carboxyalkylation of chitosan, such as N-(carboxymethyl)chitosan or N-(carboxybutyl)chitosan sold under the name “EVALSAN” by the company Jan Dekker.

(7) Polymers corresponding to the general formula (VI), for example described in French Patent FR 1,400,366, the disclosure of which is specifically incorporated by reference herein: 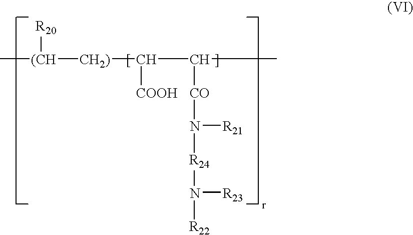
wherein:
R₂₀ represents a hydrogen atom or a CH₃O, CH₃CH₂O or phenyl radical,
R₂₁, denotes hydrogen or a lower alkyl radical, such as methyl or ethyl,
R₂₂ denotes hydrogen or a lower alkyl radical, such as methyl or ethyl, and
R₂₃ denotes a lower alkyl radical, such as methyl or ethyl, or a radical corresponding to the formula: —R₂₄—N(R₂₂)₂,
R₂₄ representing a —CH₂—CH₂—, —CH₂—CH₂—CH₂— or —CH₂—CH(CH₃)— group and
R₂₂ having the meanings mentioned above,
   and the higher homologues of these radicals containing up to 6 carbon atoms.

(8) Amphoteric polymers of the -D-X-D-X- type chosen from:
a) the polymers obtained by reacting chloroacetic acid or sodium chloroacetate with compounds containing at least one unit of formula:
   -D-X-D-X-D- (VII)
   wherein D denotes a radical 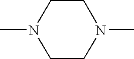
   and X denotes the E or E′ symbol,
E or E′ independently denote a divalent radical which is a straight- or branched-chain alkylene radical containing up to 7 carbon atoms in the main chain, which is unsubstituted or substituted by hydroxyl groups, and which can additionally contain oxygen, nitrogen or sulphur atoms or from 1 to 3 aromatic and/or heterocyclic rings; the oxygen, nitrogen and sulphur atoms being present in the form of ether, thioether, sulphoxide, sulphone, sulphonium, alkylamine or alkenylamine groups or hydroxyl, benzylamine, amine oxide, quaternary ammonium, amide, imide, alcohol, ester and/or urethane groups.

b) The polymers of formula:
-D-X-D-X- (VII′)
wherein D denotes a radical 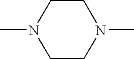
and X denotes the E or E′ symbol and at least once E′, E having the meaning indicated above in formula VII, and
E′ is a divalent radical which is a straight- or branched-chain alkylene radical having up to 7 carbon atoms in the main chain, the radical being unsubstituted or substituted by one or a number of hydroxyl radicals, and containing one or a number of nitrogen atoms, the nitrogen atom being substituted by an alkyl chain optionally interrupted by an oxygen atom and necessarily containing one or a number of carboxyl functional groups or one or a number of hydroxyl functional groups betainized by reaction with chloroacetic acid or sodium chloroacetate.

(9) (C₁-C₅)alkyl vinyl ether/maleic anhydride copolymers in which the maleic anhydride has been partially modified by semiamidification with an N,N-dialkylaminoalkylamine, such as N,N-dimethylaminopropylamine, or by semiesterification with an N,N-dialkanolamine. These copolymers can also contain other vinyl comonomers, such as vinylcaprolactam.

The particularly preferred fixing amphoteric polymers according to the invention are those from the family (2), such as the copolymers whose CTFA name is Octylacrylamide/acrylates/butylaminoethyl methacrylate copolymer, such as the products sold under the names AMPHOMER, AMHOMER LV 71 or LOVOCRYL 47 by the company National Starch and those from the family (4), such as the methyl methacrylate/methyl dimethylcarboxymethylammonioethylmethacrylate copolymer, for example sold under the name DIAFORMER Z301 by the company Sandoz.

The fixing non-ionic polymers which can be used according to the present invention are chosen, for example, from
vinylpyrrolidone homopolymers;
copolymers of vinylpyrrolidone and of vinyl acetate;
polyalkyloxazolines, such as the polyethyloxazolines provided by the company Dow Chemical under the names PEOX 50 000, PEOX 200 000 and PEOX 500 000;
vinyl acetate homopolymers, such as the product provided under the name of APPRETAN EM by the company Hoechst or the product provided under the name RHODOPAS A 012 by the company Rhône-Poulenc;
copolymers of vinyl acetate and of acrylic ester, such as the product provided under the name of RHODOPAS AD 310 from Rhône-Poulenc;
copolymers of vinyl acetate and of ethylene, such as the product provided under the name of APPRETAN TV by the company Hoechst;
copolymers of vinyl acetate and of maleic ester, for example of dibutyl maleate, such as the product provided under the name of APPRETAN MB EXTRA by the company Hoechst;
copolymers of polyethylene and of maleic anhydride;
alkyl acrylate homopolymers and alkyl methacrylate homopolymers, such as the product provided under the name MICROPEARL RQ 750 by the company Matsumoto or the product provided under the name LUHYDRAN A 848 S by the company BASF;
copolymers of acrylic esters, such as, for example, copolymers of alkyl acrylates and of alkyl methacrylates, such as the products provided by the company Rohm & Haas under the names PRIMAL AC-261 K AND EUDRAGIT NE 30 D, by the company BASF under the names ACRONAL 601 or LUHYDRAN LR 8833 or 8845 or by the company Hoechst under the names APPRETAN N 9213 or N 9212;
copolymers of acrylonitrile and of a non-ionic monomer chosen, for example, from butadiene and alkyl (meth)acrylates; mention may be made of the products provided under the names NIPOL LX 531 B by the company Nippon Zeon or those provided under the name CJ 0601 B by the company Rohm & Haas;
polyurethanes, such as the products provided under the names ACRYSOL RM 1020 or ACRYSOL RM 2020 by the company Rohm & Haas or the products URAFLEX XP 401 UZ or URAFLEX XP 402 UZ by the company DSM Resins;
copolymers of alkyl acrylate and of urethane, such as the product 8538-33 by the company National Starch;
polyamides, such as the product ESTAPOR LO 11 provided by the company Rhône-Poulenc;
chemically modified or non-modified non-ionic guar gums.

The non-modified non-ionic guar gums are, for example, the products sold under the name VIDOGUM GH 175 by the company Unipectine and under the name JAGUAR C by the company Meyhall.

The modified non-ionic guar gums which can be used according to the invention are preferably modified by C₁-C₆ hydroxyalkyl groups. Mention may be made, by way of example, of hydroxymethyl, hydroxyethyl, hydroxypropyl and hydroxybutyl groups.

These guar gums are well known in the state of the art and can, for example, be prepared by reacting the corresponding alkene oxides, such as for example propylene oxide, with guar gum, so as to obtain a guar gum modified by hydroxypropyl groups.

Such non-ionic guar gums optionally modified by hydroxyalkyl groups are, for example, sold under the trade names JAGUAR HP8, JAGUAR HP60, JAGUAR HP120, JAGUAR DC 293 and JAGUAR HP 105 by the company Meyhall or under the name GALACTASOL 4H4FD2 by the company Aqualon.

The alkyl radicals of the non-ionic polymers have from 1 to 6 carbon atoms, unless otherwise mentioned.

According to the invention, it is also possible to use fixing polymers of grafted silicone type comprising a polysiloxane portion and a portion composed of a non-silicone organic chain, one of the two portions forming the main chain of the polymer and the other being grafted onto the said main chain. These polymers are, for example, described in Patent Applications EP-A-0,412,704, EP-A-0,412 7079 EP-A-0,640,105, WO 95/00578, EP-A-0,582,152 and WO 93/23009 and U.S. Pat. No. 4,693,935, U.S. Pat. No. 4,728,571 and U.S. Pat. No. 4,972,037, the disclosures of which are specifically incorporated by reference herein. These polymers are preferably anionic or non-ionic.

Such polymers are, for example, the copolymers capable of being obtained by radical polymerization from the mixture of monomers composed of:
a) from 50 to 90% by weight of tert-butyl acrylate;
b) from 0 to 40% by weight of acrylic acid;
c) from 5 to 40% by weight of silicone macromer of formula: 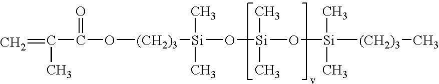
   wherein:
v is a number ranging from 5 to 700,
the percentages by weight being calculated with respect to the total weight of the monomers.

Other examples of grafted silicone polymers are in particular polydimethylsiloxanes (PDMS) on which are grafted, via a connecting link of thiopropylene type, mixed polymer units of the poly((meth)acrylic acid) type and of the poly(alkyl. (meth)acrylate) type and polydimethylsiloxanes (PDMS) on which are grafted, via a connecting link of thiopropylene type, polymer units of the poly(isobutyl (meth)acrylate) type.

According to the present invention, the fixing polymers are preferably anionic or amphoteric polymers.

The fixing anionic or amphoteric polymers can, if necessary, be partially or completely neutralized. The neutralizing agents are, for example, sodium hydroxide, potassium hydroxide, 2-amino-2-methyl-1-propanol, monoethanolamine, triethanolamine or triisopropanolamine or inorganic or organic acids, such as hydrochloric acid or citric acid.

The fixing polymer or polymers are, for example, present in concentrations ranging from 0.05% to 20% by weight and preferably in concentrations ranging from 0.1% to 10% by weight with respect to the total weight of the composition.

The amphoteric starch or starches can be present in concentrations of ranging from 0.01% to 15% by weight and preferably in concentrations ranging from 0.05% to 10% by weight and more particularly still ranging from 0.1% to 5% by weight with respect to the total weight of the composition.

The cosmetically acceptable medium is preferably composed of water or a mixture of water and of cosmetically acceptable solvents, such as monoalcohols, polyalcohols, glycol ethers or fatty acid esters, which can be used alone or as a mixture. These solvents are preferably C₁-C₆ alcohols.

Mention may be made, among these alcohols, of ethanol or isopropanol, polyalcohols, such as diethylene glycol, or glycol ethers, such as the monoalkyl ethers of glycol, of diethylene glycol, of propylene glycol or of dipropylene glycol. Ethanol is, particularly preferred.

The composition of the invention can also contain at least one additive chosen from thickeners, fatty acid esters, esters of fatty acids and of glycerol, volatile or non-volatile silicones which are soluble or insoluble in the composition, surfactants, fragrances, preservatives, sunscreening agents, proteins, vitamins, polymers, vegetable, animal, mineral or synthetic oils and any other additive conventionally used in cosmetic compositions for keratinous fibres.

These additives are present in the composition according to the invention in proportions which can range from 0 to 20% by weight with respect to the total weight of the composition. The exact amount of each additive depends on its nature and is easily determined by the person skilled in the art.

Of course, the person skilled in the art will take care to choose the possible compound or compounds to be added to the composition according to the invention so that the advantageous properties intrinsically attached to the composition in accordance with the invention are not, or not substantially, detrimentally affected by the envisaged addition.

In particular, the compositions according to the invention preferably comprise less than 10% by weight, with respect to the total weight of the composition, of C₈-C₃₀ fatty acid esters. Thus, the keratinous fibres treated with the compositions according to the invention have neither a greasy feel nor a greasy appearance and the fixing power of the composition is not reduced.

The compositions according to the invention can be provided in the form of a milk, cream or lotion which may or may not be thickened.

The compositions according to the invention can be used as rinse-out products and, preferably, as leave-in products, in particular for treating the hair, form retention of the hairstyle or hair shaping of keratinous fibres, such as the hair.

They are more particularly styling products such as fixing compositions (lacquers) and styling compositions. The lotions can be packaged in various forms, in particular in atomizers or pump-action sprays or in aerosol containers, in order to provide for application of the composition in vaporized form or in the form of a foam. Such packaging forms are indicated, for example, when it is desired to obtain a spray, a lacquer or a foam for fixing or treating the hair.

When the composition according to the invention is packaged in the form of an aerosol for the purpose of obtaining a lacquer or an aerosol foam, it comprises at least one propellant which can be chosen from volatile hydrocarbons, such as n-butane, propane, isobutane, pentane, a chlorinated and/or fluorinated hydrocarbon and their mixtures. Use may also be made, as propellant, of carbon dioxide gas, nitrous oxide, dimethyl ether, nitrogen, compressed air and their mixtures.

A further subject of the invention is a process for the cosmetic treatment of keratinous fibres, such as the hair, which comprises the application on the latter of a composition as defined above.

The compositions according to them invention are prepared according to methods well known in the state of the art. In particular, the ingredients are mixed and then packaged in an appropriate container, depending on the use envisaged.

The invention will now be illustrated more fully by means of the following examples, which should not be regarded as limiting it to the embodiments described. (In the following examples, AM means Active Material).

### EXAMPLE 1

Three hairsetting lotions were prepared with the following composition:

| FORMULATION | | | |
|---|---|---|---|
| TESTED | A (Invention) | B (Comparative) | C (Comparative) |
| STARCH*¹ | 0.5 g | 1 g | — |
| Fixing | 0.5 g | — | 1 g |
| polymer*² | | | |
| Water, q.s. for | 100 g | 100 g | 100 g |
| Fixing power | 50 | 40 | 45 |

*¹Starch modified by (2-chloroethyl)aminodipropionic acid provided by the company National Starch
*²vinylpyrrolidone/acrylic acid/lauryl methacrylate terpolymer sold under the name ACRYLIDONE LM by the company ISP

Each of these compositions was applied on washed and dried hair.

A panel of 5 experienced testers was then asked to evaluate the fixing power of each composition. The grading ranges from 0 (no fixing power) to 50 (excellent fixing power).

The composition A exhibited a fixing power superior to that of the compositions B and C which only contained one of the two compounds of the invention (either a fixing polymer or an amphoteric starch).

Hair treated with the composition (A) according to the invention also exhibited good feel properties.

### EXAMPLE 2

Three hairsetting lotions were prepared with the following composition:

| FORMULATION | | | |
|---|---|---|---|
| TESTED | A (Invention) | B (Comparative) | C (Comparative) |
| STARCH*¹ | 0.5 g | 1 g | — |
| Fixing | 0.5 g | — | 1 g |
| polymer*³ | | | |
| Water, q.s. for | 100 g | 100 g | 100 g |
| Fixing power | 35 | 30 | 30 |

*¹Starch modified by (2-chloroethyl)aminodipropionic acid provided by the company National Starch
*³Acrylamide/acrylic acid/dimethyldiallylammonium chloride terpolymer as a 10% aqueous solution sold under the name MERQUAT PLUS 3330 by the company Calgon

Each of these compositions was applied on washed and dried slightly bleached hair.

A panel of 5 experienced testers was then asked to evaluate the fixing power of each composition. The grading ranges from 0 (no fixing power) to 50 (excellent fixing power).

The composition A exhibited a fixing power superior to that of the compositions B and C which only contained one of the two compounds of the invention.

Hair treated with the composition (A) according to the invention also exhibited good feel, softness and disentangling properties.

### EXAMPLE 3

A fixing spray composition packaged in a pump-action spray was prepared with the following composition:

| | |
|---|---|
| Starch modified by (2-chloroethyl)-aminodipropionic | 0.8 g |
| acid | |
| Acrylamide/acrylic acid/dimethyldiallylammonium | 0.2 gAM |
| chloride terpolymer as a 10% aqueous solution, sold | |
| under the name MERQUAT PLUS 3330 by the | |
| company Calgon | |
| Polyquaternium-37 (INCO name), sold under the name | 0.6 gAM |
| SALCARE SC 95 by the company Allied Colloid | |
| Water q.s. for | 100 g |

The composition exhibited the same properties as those of Example 1.

### EXAMPLE 4

A styling gel composition was prepared with the following composition:

| | |
|---|---|
| Starch modified by (2-chloroethyl)-aminodipropionic | 0.5 g |
| acid | |
| Acrylamide/acrylic acid/dimethyldiallylammonium | 0.5 gAM |
| chloride terpolymer as a 10% aqueous solution, sold | |
| under the name MERQUAT PLUS 3330 by the | |
| company Calgon | |
| Crosslinked poly(acrylic acid), sold under the name | 0.6 gAM |
| SYNTHALEN K by the company 3V | |
| 95° Ethanol | 8.5 g |
| Triethanolamine q.s. | pH 7.5 |
| Water q.s. for | 100 g |

The composition was applied on washed and towel-dried hair. It resulted in good form retention of the hairstyle and in good disentangling and feel properties.

### EXAMPLE 5

A fixing milk packaged in a pump-action spray was prepared with the following composition:

| | |
|---|---|
| Vinylpyrrolidone/acrylic acid/lauryl methacrylate | 2 gAM |
| terpolymer, sold under the name ACRYLIDONE LM by | |
| the company ISP | |
| 2-Amino-2-methyl-1-propanol | 0.6 g |
| Starch modified by (2-chloroethyl)-aminodipropionic | 0.5 g |
| acid | |
| Water q.s. for | 100 g |

The composition was applied on washed and towel-dried hair. It resulted in good form retention of the hairstyle and in good disentangling and feel properties.

### EXAMPLE 6

A styling care gel composition was prepared with the following composition:

| | |
|---|---|
| Starch modified by (2-chloroethyl)-aminodipropionic | 0.5 g |
| acid | |
| Hydroxyethyl cellulose/diallyldimethylammonium | 0.3 gAM |
| chloride copolymer, sold under the name CELQUAT L | |
| 200 by the company National Starch | |
| Hydroxypropyl guar gum, sold by the company | 0.3 g |
| Rhône-Poulenc under the name JAGUAR HP 105 | |
| Crosslinked poly(acrylic acid), sold under the name | 0.4 gAM |
| SYNTHALEN K by the company 3V | |
| 95° Ethanol | 8.5 g |
| 2-Amino-2-methyl-1-propanol q.s. | pH 7.5 |
| Water q.s. for | 100 g |

The composition was applied on washed and towel-dried hair. It resulted in good form retention of the hairstyle and in good disentangling and feel properties.

1-30. (canceled)

31. A cosmetic composition for the treatment of keratinous fibres comprising, in a cosmetically acceptable medium, at least one anionic fixing polymer and at least one amphoteric starch.

32. The cosmetic composition for the treatment of keratinous fibres according to claim 31, wherein the at least one anionic fixing polymer is present in an amount ranging from 0.05 to 20% by weight, relative to the total weight of the composition.

33. The cosmetic composition for the treatment of keratinous fibres according to claim 32, wherein the at least one anionic fixing polymer is present in an amount ranging from 0.1 to 10% by weight, relative to the total weight of the composition.

34. The cosmetic composition for the treatment of keratinous fibres according claim 31, wherein the at least one amphoteric starch is present in an amount ranging from 0.01 to 15% by weight, relative to the total weight of the composition.

35. The cosmetic composition for the treatment of keratinous fibres according to claim 34, wherein the at least one amphoteric starch is present in an amount ranging from 0.05 to 10% by weight, relative to the total weight of the composition.

36. The cosmetic composition for the treatment of keratinous fibres according to claim 31, wherein the at least one anionic fixing polymer is chosen from:
polymers comprising carboxyl units derived from at least one monomer chosen from monomers of formula II and salts thereof: 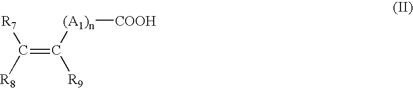
wherein:
n is an integer ranging from 0 to 10;
A₁ is a methylene group, optionally connected to the carbon atom of the unsaturated group or to the neighboring methylene group, when n is greater than 1, via a heteroatom;
R₇ is chosen from a hydrogen atom and phenyl and benzyl groups;
R₈ is chosen from a hydrogen atom and lower alkyl and carboxyl groups; and
R₉ is chosen from a hydrogen atom, lower alkyl groups, and —CH₂—COOH, phenyl, and benzyl groups; and
polymers comprising units derived from sulphonic acid.

37. The cosmetic composition for the treatment of keratinous fibres according to claim 36, wherein the heteroatom of A₁ is chosen from oxygen and sulphur.

38. The cosmetic composition for the treatment of keratinous fibres according to claim 36, wherein the units derived from sulphonic acid are chosen from vinylsulpho, styrenesulpho, napthalenesulpho, and acrylamidoalkylsulpho units.

39. The cosmetic composition for the treatment of keratinous fibres according to claim 31, wherein the at least one anionic fixing polymer is chosen from:
A) homopolymers and copolymers derived from acrylic and methacrylic acid and the salts thereof, copolymers derived from acrylic acid and acrylamide and the salts thereof and the sodium salts of polyhydroxycarboxylic acids, wherein the acrylic acid copolymers differ from those defined below in C);
B) copolymers derived from crotonic acid, wherein the copolymers are optionally grafted and crosslinked;
C) copolymers derived from mono-unsaturated C₄-C₈ carboxylic acids and anhydrides chosen from:
copolymers derived from (i) at least one monomer chosen from maleic, fumaric and itaconic acids and anhydrides and (ii) at least one monomer chosen from vinyl esters, vinyl ethers, vinyl halides, phenylvinyl derivatives, acrylic acid and the esters thereof, and wherein the anhydride functional groups of the copolymers are optionally monoesterified or monoamidified;
copolymers derived from (i) at least one monomer chosen from maleic, citraconic and itaconic anhydrides and (ii) at least one monomer chosen from allyl and methallyl esters, optionally comprising at least one group chosen from acrylamide, methacrylamide, α-olefin, acrylic ester, methacrylic ester, acrylic acid, methacrylic acid, and vinylpyrrolidone groups, in their chain, and wherein the anhydride functional groups of these copolymers are optionally monoesterified or monoamidified; and
D) polyacrylamides comprising carboxylate groups.

40. The cosmetic composition for the treatment of keratinous fibres according to claim 31, wherein the at least one anionic fixing polymer is chosen from
copolymers derived from acrylic acid or methacrylic acid and at least one monoethylenic monomer, optionally grafted onto a polyalkylene glycol, and optionally crosslinked, and
copolymers derived from acrylic acid or methacrylic acid and at least one monoethylenic monomer comprising, in their chain, at least one unit chosen from optionally N-alkylated and N-hydroxyalkylated acrylamide units.

41. The cosmetic composition for the treatment of keratinous fibres according to claim 40, wherein the polyalkylene glycol is polyethylene glycol.

42. The cosmetic composition for the treatment of keratinous fibres according to claim 40, wherein the at least one monoethylenic monomer is chosen from ethylene, styrene, vinyl esters and esters of acrylic and methacrylic acid,

43. The cosmetic composition for the treatment of keratinous fibres according to claim 40, wherein the copolymers derived from acrylic acid or methacrylic acid and at least one monoethylenic monomer are chosen from copolymers derived from acrylic acid and C₁-C₄ alkyl methacrylate.

44. The cosmetic composition for the treatment of keratinous fibres according to claim 39, wherein the copolymers derived from crotonic acid comprise in their chain, at least one unit chosen from vinyl acetate and propionate units and optionally other monomers.

45. The cosmetic composition for the treatment of keratinous fibres according to claim 44, wherein the optional other monomers are chosen from allyl and methallyl esters, vinyl ethers and vinyl esters of a linear or branched saturated carboxylic acid comprising at least one long hydrocarbon chain.

46. The cosmetic composition for the treatment of keratinous fibres according to claim 45, wherein the at least one long hydrocarbon chain comprises at least 5 carbon atoms.

47. The cosmetic composition for the treatment of keratinous fibres according to claim 39, wherein the at least one anionic fixing polymer is chosen from:
copolymers derived from acrylic acid;
copolymers derived from crotonic acid;
copolymers derived from (i) at least one monomer chosen from maleic, fumaric and itaconic acids and anhydrides and (ii) at least one monomer chosen from vinyl esters, vinyl ethers, vinyl halides, phenylvinyl derivatives, and acrylic acid and the esters thereof;
copolymers derived from methacrylic acid and methyl methacrylate;
copolymers derived from methacrylic acid and ethyl acrylate;
copolymers derived from vinyl acetate and crotonic acid; and
copolymers derived from vinyl acetate and crotonic acid, grafted with a polyethylene glycol.

48. The cosmetic composition for the treatment of keratinous fibres according to claim 39, wherein the at least one anionic fixing polymer is chosen from:
terpolymers derived from acrylic acid, ethyl acrylate and N-tert-butylacrylamide;
terpolymers derived from vinyl acetate, vinyl tert-butylbenzoate and crotonic acid,
terpolymers derived from crotonic acid, vinyl acetate and vinyl neododecanoate,
copolymers derived from monoesterified maleic anhydride and methyl vinyl ether,
copolymers derived from methacrylic acid and methyl methacrylate,
copolymers derived from methacrylic acid and ethyl acrylate, and
terpolymers derived from vinylpyrrolidone, acrylic acid, lauryl methacrylate.

49. The cosmetic composition for the treatment of keratinous fibres according to claim 31, wherein the at least one amphoteric starch is chosen from the compounds of formulae (I) to (IV): 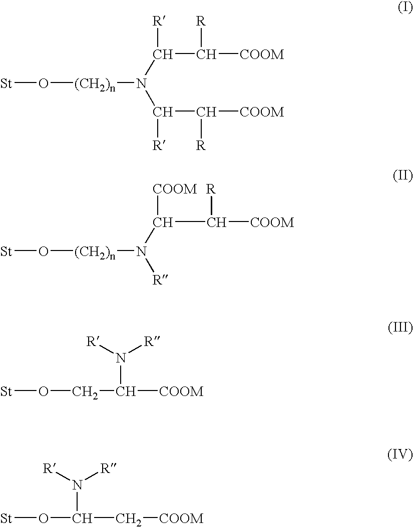
wherein:
St—O is a starch molecule;
R, which may be identical or different, is chosen from a hydrogen atom and a methyl radical;
R′, which may be identical or different, is chosen from a hydrogen atom, a methyl radical and a —COOH group;
n is an integer equal to 2 or 3;
M, which may be identical or different, is chosen from a hydrogen atom, alkali and alkaline-earth metals, NH₄, and organic amines; and
R″, which may be identical or different, is chosen from a hydrogen atom and alkyl radicals comprising from 1 to 18 carbon atoms.

50. The cosmetic composition for the treatment of keratinous fibres according to claim 49, wherein the at least one amphoteric starch is chosen from the compounds of formulae (I) and (II).

51. The cosmetic composition for the treatment of keratinous fibres according to claim 49, wherein R, R′, R″ and M are each a hydrogen atom and n is equal to 2.

52. The cosmetic composition for the treatment of keratinous fibres according to claim 31, wherein the cosmetically acceptable medium is chosen from water and mixtures of water and at least one cosmetically acceptable solvent.

53. A process for the cosmetic treatment of keratinous fibres comprising applying to the fibres at least one care, styling, or fixing composition comprising, in a cosmetically acceptable medium, at least one anionic fixing polymer and at least one amphoteric starch.

54. The process according to claim 53, wherein the keratinous fibres are hair.

55. A cosmetic composition comprising, in a cosmetically acceptable medium, at least one anionic fixing polymer and at least one amphoteric starch, wherein the cosmetic composition is provided in a form chosen from care compositions, styling compositions, and fixing compositions.

56. A process for making a cosmetic composition for the treatment of keratinous fibres comprising including in the composition, in a cosmetically acceptable medium, at least one anionic fixing polymer and at least one amphoteric starch.
